(19) United States
(12) Patent Application Publication
(10) Pub. No.: US 2026/0227400 A1
(43) Pub. Date: Aug. 6, 2026

(54) SARS-CoV-2 DETECTION METHOD AND KIT THEREFOR

(71) Applicant: Denka Company Limited, Tokyo (JP)

(72) Inventors: Toru YAMAGUCHI, Gosen-shi (JP); Daisuke KATO, Gosen-shi (JP); Shino MURAMATSU, Gosen-shi (JP)

(73) Assignee: Denka Company Limited, Tokyo (JP)

(21) Appl. No.: 19/151,683

(22) PCT Filed: Feb. 7, 2024
(86) PCT No.: PCT/JP2024/003986

§ 371 Date: Jul. 29, 2025

(30) Foreign Application Priority Data
Feb. 8, 2023 (JP) 2023-017650

(51) Int. Cl. G01N 33/569; C07K 16/104

(52) U.S. Cl. CPC G01N 33/56983; C07K 16/104; C07K 2317/34; G01N 2333/165; G01N 2469/10; G01N 2470/04

(57) ABSTRACT

Disclosed is a high performance anti-SARS-CoV-2 antibody and a detection reagent using it. The method of detecting SARS-CoV-2 includes detecting SARS-CoV-2 in a sample by an immunoassay method using a monoclonal antibody that specifically reacts with 301aa-319aa, 301aa-320aa, or 351aa-375aa of the amino acid sequence of the N protein of SARS-CoV-2, or an antigen-binding fragment thereof. The detection kit for SARS-CoV-2 includes a monoclonal antibody that specifically reacts with 301aa-319aa, 301aa-320aa, or 351aa-375aa of the amino acid sequence of the N protein of SARS-CoV-2, or an antigen-binding fragment thereof.

### TECHNICAL FIELD

The present invention relates to a method of detecting SARS-CoV-2 and a kit therefor.

### BACKGROUND ART

SARS-CoV-2 is a virus belonging to the Coronaviridae family, having a single-stranded positive-sense RNA viral genome. In March 2020, the infectious disease (COVID-19) caused by SARS-CoV-2 became a pandemic, resulting in many infections worldwide. To date in 2022, vaccines have been developed in various countries, and vaccination is progressing globally, but due to the emergence of mutant strains, there is no clear end in sight for the pandemic.

COVID-19 is a respiratory disease, and 80% of patients recover with mild symptoms. On the other hand, if the disease becomes severe, it leads to dyspnea, and treatment is performed using oxygen inhalation or ECMO. According to the materials from the Ministry of Health, Labor and Welfare's Advisory Board on Countermeasures against Novel Coronavirus Infections announced on Sep. 7, 2022, the case fatality rate of COVID-19 in Japan during the 6th wave was 2.67% for those in their 80s and 4.05% for those in their 90s, making early medical intervention for high-risk individuals important.

COVID-19 infection is diagnosed using PCR methods and antigen test methods for SARS-CoV-2. As a method for rapidly and easily detecting SARS-CoV-2, immunochromatography methods using anti-SARS-CoV-2 antibodies have been developed, but with no end in sight for the pandemic, more accurate tests are required.

### PRIOR ART DOCUMENT

### Patent Document

PATENT DOCUMENT 1 WO 2021/181994

### SUMMARY OF INVENTION

### Problem which the Invention Tries to Solve

Currently, various test reagents using various anti-SARS-CoV-2 antibodies are commercially available. However, when SARS-CoV-2 was detected with these conventional SARS-CoV-2 test reagents, their performance was not sufficient.

In view of the above situation, an object of the present invention is to provide a high-performance anti-SARS-CoV-2 antibody and a test reagent using the same.

### Means for Solving the Problem

As a result of diligent research, the present inventors have found that the performance of detecting SARS-CoV-2 can be improved by using an antibody against a specific region of the N protein (nucleoprotein, nucleocapsid protein) of SARS-CoV-2 in a sample, and have completed the present invention.

That is, the present invention provides the following. (1) A method of detecting SARS-CoV-2, comprising detecting SARS-CoV-2 in a sample by an immunoassay method using a monoclonal antibody that specifically reacts with 301aa to 319aa, 301aa to 320aa, or 351aa to 375aa of the amino acid sequence of the N protein of SARS-CoV-2, or an antigen-binding fragment thereof. (2) The method according to (1), wherein the immunoassay method is an immunochromatography method. (3) A detection kit for SARS-CoV-2, comprising a monoclonal antibody that specifically reacts with 301aa to 319aa, 301 aa to 320aa, or 351aa to 375aa of the amino acid sequence of the N protein of SARS-CoV-2, or an antigen-binding fragment thereof. (4) The detection kit according to (3), which is an immunochromatographic test strip.

### Effects of Invention

The method of the present invention has high sensitivity because it uses an antibody that reacts with a specific region of the N protein of SARS-CoV-2 for immunoassay. The present invention also provides a detection kit that uses the novel detection method of the present invention.

### MODE FOR CARRYING OUT THE INVENTION

The method of the present invention comprises detecting SARS-CoV-2 in a sample by an immunoassay method using a monoclonal antibody that specifically reacts with 301aa to 319aa, 301aa to 320aa, or 351aa to 375aa of the amino acid sequence of the N protein of SARS-CoV-2, or an antigen-binding fragment thereof. Herein, “specifically react” means to undergo an antigen-antibody reaction. Therefore, in a system where a protein and the antibody are mixed, the antibody does not cause an antigen-antibody reaction at a detectable level with proteins other than the target protein component in the antigen, or even if the coexisting substances other than the antigen cause some kind of binding reaction or association reaction with the antibody, only a reaction that is clearly weaker than the antigen-antibody reaction of the antibody with the antigen occurs. The amino acid sequence of the N protein of SARS-CoV-2 is shown by SEQ ID NO: 1. The amino acid sequence shown by SEQ ID NO: 1 is the amino acid sequence of the wild-type strain (Wuhan strain), but the present invention is not limited to the wild-type strain and is also applicable to other strains derived from the wild-type strain.

Antigen-binding fragments, in which only the antigen-binding site is isolated based on the monoclonal antibody used in the method of the present invention, can also be used in the method of the present invention. That is, the scope of the present invention also includes cases of using a fragment having specific antigen-binding properties (antigen-binding fragment) which binds to the specific region of N protein of SARS-CoV-2, prepared by a known method, such as Fab, Fab′, F(ab′)₂, single-chain antibody (scFv), recombinant antibody, or modified antibody (e.g., chimeric antibody, humanized antibody, human antibody, CDR-grafted antibody, primatized antibody, deimmunized antibody, synhumanized antibody, dsFv, diabody, minibody). The class of the monoclonal antibody is not limited to IgG, and may be IgM or IgY.

The monoclonal antibody used in the method of the present invention can be obtained by immunizing an animal to be immunized with a polypeptide consisting of the corresponding epitope, or a polypeptide, complex, or extract containing the epitope, using a known immunological technique, and preparing a hybridoma by a conventional method using cells from the immunized animal. The immunogen can be obtained from a culture medium, but it can also be obtained by incorporating DNA encoding an arbitrary antigen into a plasmid vector and introducing this into a host cell for expression. An arbitrary antigen or its partial peptide to be used as an immunogen can also be expressed as a fusion protein with proteins exemplified below, and used as an immunogen after purification or without purification. For the production of fusion proteins, glutathione S-transferase (GST), maltose-binding protein (MBP), thioredoxin (TRX), Nus tag, S tag, HSV tag, FRAG tag, polyhistidine tag, etc., which are generally used by those skilled in the art as “protein expression/purification tags”, can be utilized. It is preferable to use these fusion proteins as immunogens after cleaving the arbitrary antigen or its partial peptide portion and the other tag portion using a digestive enzyme, followed by separation and purification.

Preparation of monoclonal antibodies from immunized animals can be easily performed by the well-known method of Köhler et al. (Kohler et al., Nature, vol. 256, p 495-497 (1975)). That is, antibody-producing cells such as spleen cells or lymphocytes are collected from the immunized animal, fused with mouse myeloma cells by a conventional method to produce hybridomas, the obtained hybridomas are cloned by a method such as limiting dilution, and among the monoclonal antibodies produced by each cloned hybridoma, monoclonal antibodies that undergo an antigen-antibody reaction with the antigen used for immunizing the animal are selected.

Purification of monoclonal antibodies from ascites fluid or culture supernatant can be performed using known immunoglobulin purification methods. Examples include fractionation methods by salting out using ammonium sulfate or sodium sulfate, PEG fractionation methods, ethanol fractionation methods, DEAE ion-exchange chromatography methods, and gel filtration methods. Purification is also possible by affinity chromatography using a carrier to which Protein A, Protein G, or Protein L is bound, depending on the animal species immunized and the class of the monoclonal antibody.

Moreover, the monoclonal antibody used in the present invention can be produced by genetically modified plants. Such antibodies can be produced using a plant transient expression system.

The monoclonal antibody used in the present invention can also be obtained as a recombinant product using mammalian cells as the expression host. Examples of mammalian cells in this case include CHO (Chinese Hamster Ovary) cells and HEK293 (Human Embryonic Kidney cells 293) cells, but are not limited thereto. Furthermore, regarding the method for obtaining it as a recombinant product, examples thereof include transient expression systems using plasmid vectors or viral vectors lacking autonomous replication ability, semi-stable expression systems using episomal vectors provided with a nuclear localization signal and having autonomous replication ability, and stable expression systems in which the target gene is inserted into the genome of the expression host, and are not particularly limited.

The N protein of SARS-CoV-2 (nucleoprotein, nucleocapsid protein; hereinafter also simply referred to as “N protein” in this specification) consists of an amino acid sequence of 419 amino acids (see SEQ ID NO: 1). As described above, the monoclonal antibody used in the present invention specifically reacts with 301aa to 319aa, 301aa to 320aa, or 351aa to 375aa within the N protein of SARS-CoV-2. Note that “aa” indicates the position of the amino acid residue counted from the N-terminus of the polypeptide. Therefore, for example, 301aa in the N protein means the 301st amino acid residue counted from the N-terminus of the N protein. Whether or not a certain monoclonal antibody specifically reacts with these regions can be known by examining by immunoassay whether or not the polypeptide consisting of each region and the monoclonal antibody undergo antigen-antibody reaction.

The antibody of the present invention that specifically reacts with a specific region in the N protein of anti-SARS-CoV-2 has flexibility in its reactivity and may, for example, recognize a conformational structure formed by these peptides.

In the immunoassay method of the present invention, measurement is performed by an immunoassay utilizing an antigen-antibody reaction between a monoclonal antibody prepared as described above or an antigen-binding fragment thereof (hereinafter, in the description up to the Examples, “antibody” means “antibody or an antigen-binding fragment thereof” unless it clearly dictates otherwise from the context) and an antigen in a sample. As the immunoassay method for this purpose, any method known to those skilled in the art can be used, such as a competitive method, an agglutination method, a Western blot method, an immunostaining method, and a sandwich method. In the present invention, “measurement” includes quantification, semi-quantification, and detection.

As the immunoassay method of the present invention, the sandwich method is preferred. In the sandwich method, a complex is formed by sandwiching the antigen with two antibodies, and the complex is detected. The sandwich method itself is well-known in the field of immunoassay, and can be performed, for example, by immunochromatography or ELISA methods. These sandwich methods themselves are all well known, and the method of the present invention can be performed by a well-known sandwich method, except for using a monoclonal antibody that recognizes the hydrophobic region of the N protein described above as an antigen.

In the sandwich method, one or two or more types of antibodies (an antibody immobilized on a solid phase and a labeled antibody) that recognize the antigen are used. When two or more types of antibodies are used, at least one of these two types of antibodies is a monoclonal antibody that recognizes the above-mentioned specific region in the N protein of anti-SARS-CoV-2 as an antigen. Also, a complex may be formed by sandwiching the antigen with the same two antibodies. Preferably, one or two types of antibodies that recognize the above peptide are used.

In immunoassays based on the sandwich method detection principle, as the solid phase on which the antibody is immobilized, anything that can immobilize the antibody by known techniques can be used, and for example, known materials such as porous thin films (membranes) having capillary action, particulate matter, test tubes, and resin plates can be arbitrarily selected. As the substance for labeling the antibody, enzymes, radioisotopes, fluorescent substances, luminescent substances, colored particles, colloidal particles, etc., can be used. Among the immunoassay methods using the various materials mentioned above, the immunochromatography method, which is a lateral flow type immunoassay method using a membrane, is particularly preferred from the viewpoint of simplicity and rapidity of clinical testing.

The present invention also provides a detection kit for SARS-CoV-2, comprising a monoclonal antibody that specifically reacts with 301aa to 319aa, 301aa to 320aa, or 351aa to 375aa of the amino acid sequence of the N protein of SARS-CoV-2. As such a kit, an immunoassay device capable of performing an immunoassay in a lateral flow format is particularly preferable. The immunochromatographic test strip provided by the present invention is an immunoassay device comprising a support having a detection region on which an antibody for capturing a measurement target (antigen) (antibody 1) is immobilized, a label region having a movable labeled antibody (antibody 2), a sample pad for dropping a sample, an absorption pad for absorbing the developed sample solution, and a backing sheet for laminating these members together, wherein at least one of antibody 1 and antibody 2 is a monoclonal antibody that specifically reacts with the above-mentioned specific region in the N protein of anti-SARS-CoV-2. This immunoassay device is also called an immunochromatographic test strip. Note that multiple monoclonal antibodies can be used for the support having a detection region on which an antibody (antibody 1) capturing the analyte (antigen) is immobilized, and as the movable labeled antibody (antibody 2). Also, a polyclonal antibody can be combined. The kit may further include a brochure, a sample collection tool, etc.

The support is a material having the ability to immobilize the antibody for capturing the substance to be detected (antigen), and also having the ability not to hinder the horizontal passage of liquid. Preferably, it is a porous thin film having capillary action, and is a material capable of transporting liquid and components dispersed therein by absorption. The material constituting the support is not particularly limited, and examples include cellulose, nitrocellulose, cellulose acetate, polyvinylidene difluoride (PVDF), glass fiber, nylon, polyketone, etc. Among these, those made into a thin film using nitrocellulose are more preferable. A membrane on which an antibody is immobilized is called an antibody-immobilized membrane.

The label region is composed of a porous substrate containing a labeled antibody, and commonly used materials such as glass fiber or non-woven fabric can be used as the substrate material. The substrate is preferably in the form of a pad with a thickness of about 0.3 mm to 0.6 mm in order to impregnate a large amount of labeled antibody. The porous substrate impregnated with the labeled antibody and dried is also called a dry pad.

For labeling the labeled antibody, enzymes such as alkaline phosphatase and horseradish peroxidase, metal colloids such as gold colloid, silica particles, cellulose particles, colored polystyrene particles, and colored latex particles are often used. When using colored particles such as metal colloid particles, colored polystyrene particles, or colored latex particles, coloring occurs due to the aggregation of these labeling reagents, so this coloring is measured. Particles on which antibodies are immobilized are called antibody-immobilized particles. The amount of antibody immobilized is not particularly limited, but it is sufficient if several ng to several tens of μg are present in the labeling region.

The detection region refers to a part of the support where the antibody that captures the substance to be detected (antigen) is immobilized. The detection region is provided with at least one region where the antibody for capturing the antigen is immobilized. The detection region only needs to be included in the support, and the antibody should be immobilized on the support. The amount of the antibody immobilized is not particularly limited, but it is sufficient if several ng to several tens of μg are immobilized in the detection region.

The sample pad is a site for applying the sample and is a porous material. The sample pad is the most upstream site of the immunoassay device. Common filter paper, glass fiber, non-woven fabric, etc., can be used for this material. To use a large amount of sample for immunoassay, it is preferably in the form of a pad with a thickness of about 0.3 mm to 1 mm. The sample also includes samples prepared using the specimen, such as samples obtained by suspending the specimen in another solution.

The absorption zone is a member for absorbing components supplied to the support that were not involved in the reaction in the detection region. For this material, highly water-retentive filter paper, sponge, etc., made of a common natural polymer compound, synthetic polymer compound, etc., can be used, but those with high water absorbency are preferable for promoting the development of the sample.

The backing sheet is a member to which all the aforementioned materials, namely the support, sample pad, label region, absorption zone, etc., are attached and fixed with partial overlap. The backing sheet is not necessarily required if these materials can be arranged and fixed at optimal intervals, but it is generally preferable to use it for convenience in manufacturing or use.

The immunoassay device of the present invention may further include a control display region (member). The control display region is a site indicating that the test was performed correctly. For example, the control display region exists downstream of the detection region, and emits a signal by coloring etc. when the sample reaches the control display region after passing through the detection region. In the control display region, a substance that binds to the antibody bound to the labeled carrier may be immobilized, or a reagent such as a pH indicator that changes color when the sample arrives may be immobilized. When the antibody bound to the labeled carrier is a mouse monoclonal antibody, an anti-mouse IgG antibody may be used.

The size of the immunoassay device is not limited, but for example, it is about several cm to ten-odd cm in length and several mm to several cm in width.

The immunoassay device of the present invention may be housed in a storage container, which can prevent deterioration due to, for example, ultraviolet rays or moisture in the air. Also, when using contaminated or infectious samples, the storage container can prevent the testing personnel performing the assay from being contaminated or infected. For example, a resin case of an appropriate size can be used as the storage container, and the device of the present invention can be stored in the case. The storage container and the immunochromatographic test strip housed therein may be collectively referred to as an immunoassay device.

In the method of the present invention using the immunoassay device, a complex of antibody 2, which can bind to the substance to be detected (labeled reagent) labeled with a suitable labeling substance such as colored polystyrene particles or gold colloid, and the substance to be detected is developed and moved on a solid phase support on which antibody 1 is immobilized, utilizing capillary action. As a result, a complex of immobilized substance—substance to be detected—labeled reagent is formed on the solid phase support, and by detecting the signal of the labeled reagent emitted from the complex (in the case of gold colloid, the solid phase support portion where the substance capable of binding to the substance to be detected is immobilized turns red), the substance to be detected can be detected. The immunoassay method can be performed at 5-35° C., preferably at room temperature.

Note that the number of detection regions and the types of labeled antibodies included in the label region are not limited to one, and by using antibodies corresponding to multiple analytes, two or more types of antigens can be detected with the same immunoassay device.

By the method of the present invention, it is possible to detect whether or not a subject is infected with SARS-CoV-2, and when the N protein is detected in the subject sample, it can be determined that the subject is infected with SARS-CoV-2.

When the antibody that specifically reacts with the above-mentioned specific region in the N protein of the present invention is used, it is possible to specifically recognize SARS-CoV-2, and the antibody that recognizes the above-mentioned specific region in the N protein does not recognize other viruses, such as Adenovirus, Coxsackievirus, Echo virus, Herpes simplex virus, Human Metapneumovirus, Influenza virus, Measles virus, Mumps virus, Parainfluenza virus, RS virus (Respiratory syncytial virus), etc., and does not erroneously detect these viruses.

Even a clinical isolate that cannot be detected when an antibody that recognizes a region other than the Dimerization domain of the N protein of SARS-CoV-2 is used can be detected when the antibody that recognizes the above-mentioned specific region in the N protein of SARS-CoV-2 of the present invention is used.

Examples of the test sample include biological samples that may contain SARS-COV-2 proteins, such as body fluids like human or animal blood, serum, plasma, urine, semen, cerebrospinal fluid, saliva, sweat, tears, ascites, or amniotic fluid; mucus; feces; organs such as blood vessels or liver; tissues; cells, or extracts thereof. Preferred are cells and secretions from the oral cavity, tonsils, nasal cavity, pharynx, larynx, trachea, bronchi, or lungs, which are easy to collect, nasal swabs, pharyngeal swabs, gargle solutions, sputum, tracheal aspirates, bronchoalveolar lavage fluid, saliva, etc. The method for collecting these samples is not particularly limited, and known methods can be adopted. Specifically, methods using cotton swabs can be mentioned.

Hereinafter, the present invention will be described more specifically based on Examples. However, the present invention is not limited to the following Examples.

### EXAMPLES

### Example 1: Preparation of Monoclonal Antibody that Recognizes SARS-CoV-2 N Protein

### 1. Preparation of SARS-CoV-2 N Protein Antigen

A product obtained by expressing DNA encoding the SARS-CoV-2 N protein in E. coli using an expression vector, culturing for several days, and then purifying the protein was used.

### 2. Preparation of Anti-SARS-CoV-2 N Protein Monoclonal Antibody

A plurality of hybridoma cell lines producing an anti-SARS-CoV-2 N protein antibody were obtained by the “mouse iliac lymph node method” (Sado Y et al., Acta Histochem. Cytochem. 39: 89-94 (2006)), in which the SARS-CoV-2 N protein antigen of 1. was used to immunize BALB/c mice, and the iliac lymph nodes were excised from the mice that had been bred for a certain period.

The obtained cell lines were intraperitoneally administered to pristane-treated BALB/c mice, and after about 2 weeks, antibody-containing ascites were collected. From the obtained ascites, IgG was purified by affinity chromatography using a Protein A column to obtain a plurality of purified anti-SARS-CoV-2 N protein monoclonal antibodies (hereinafter sometimes referred to as “anti-N protein antibody”).

In the following Examples, antibodies selected from the plurality of obtained anti-SARS-CoV-2 N protein monoclonal antibodies in consideration of reactivity and specificity were used.

### Example 2: Analysis of Binding Region of Anti-N Protein Antibody

The binding site of the anti-N protein antibody prepared in Example 1 was investigated. The sequences to which monoclonal antibodies No. A1, A8, A14, A18, A20, A22, A23, A24, A25, A27, and A34 bind were specifically determined as follows.

Partial chains consisting of 5, 25, or 50 consecutive amino acids of the SARS-CoV-2 N protein antigen were immobilized on a plate, and the reactivity with the anti-N protein antibody obtained in Example 1 was evaluated by ELISA method. The partial chain that showed reactivity with the anti-N protein antibody was designated as the binding site of the anti-N protein antibody.

The sequences to which monoclonal antibodies No. A2, A3, A4, A5, A6, A7, A9, A10, A11, A12, A13, A15, A16, A17, A19, A21, A26, A28, A29, A30, A31, A32, A33, A35, A36, A37, and A38 bind were specifically determined as follows. Partial chains consisting of 15 to 25 consecutive amino acids of the SARS-CoV-2 N protein antigen were immobilized on microbeads, and the reactivity with the anti-N protein antibody obtained in Example 1 was evaluated using xMAP (registered trademark) (Luminex (registered trademark)). The partial chain that showed reactivity with the anti-N protein antibody was designated as the binding site of the anti-N protein antibody.

As a result, monoclonal antibodies that bind to the following sequences of the 419 amino acid sequences of the N protein of SARS-CoV-2 were obtained.

TABLE 1

| Monoclonal | |
|---|---|
| Antibody No. | Sequence |
| A1 | 1~50 |
| A2 | 3~28 |
| A3 | 6~24 |
| A4 | 22~42 |
| A5 | 22~43 |
| A6 | 23~42 |
| A7 | 60~81 |
| A8 | 76~100 |
| A9 | 88~106 |
| A10 | 88~107 |
| A11 | 88~108 |
| A12 | 90~106 |
| A13 | 90~107 |
| A14 | 101~125 |
| A15 | 114~131 |
| A16 | 114~135 |
| A17 | 114~136 |
| A18 | 126~150 |
| A19 | 155~175 |
| A20 | 156~160 |
| A21 | 156~174 |
| A22 | 161~165 |
| A23 | 166~170 |
| A24 | 171~175 |
| A25 | 176~200 |
| A26 | 185~207 |
| A27 | 226~250 |
| A28 | 266~282 |
| A29 | 301~319 |
| A30 | 301~320 |
| A31 | 326~369 |
| A32 | 337~369 |
| A33 | 344~364 |
| A34 | 351~375 |
| A35 | 366~389 |
| A36 | 369~389 |
| A37 | 372~389 |
| A38 | 376~419 |

Since the Dimerization domain of the N protein refers to the amino acid sequence in the range of amino acids 258 to 361, it was found that No. A28 to A34 are antibodies that almost recognize the Dimerization domain of the N protein.

### Example 3: Immunoassay Device for Measuring SARS-CoV-2

1. Immobilization of Anti-SARS-CoV-2 N Protein Antibody onto Nitrocellulose Membrane

A solution prepared by diluting the anti-N protein antibody separated in Example 2 with a buffer and an anti-mouse IgG antibody were provided, and the anti-N protein antibody was applied in a line on the sample pad side and the anti-mouse IgG antibody was applied in a line on the absorption pad side of a nitrocellulose membrane backed with a PET film. Thereafter, the nitrocellulose membrane was sufficiently dried under warm air to obtain an anti-N protein antibody-immobilized membrane.

2. Immobilization of Anti-SARS-CoV-2 N Protein Antibody onto Colored Polystyrene Particles

The anti-N protein antibody prepared in Example 1 was bound to colored polystyrene particles, suspended in a buffer, and sufficiently dispersed by sonication to obtain anti-N protein antibody-bound colored polystyrene particles. In this specification, these are referred to as anti-N protein antibody-immobilized particles.

### 3. Application and Drying of Anti-SARS-CoV-2 N Protein Antibody-Bound Colored Polystyrene Particles

A prescribed amount of the anti-N protein antibody-immobilized particles prepared in 2. was applied to a glass fiber non-woven fabric and sufficiently dried under warm air. In this specification, this is referred to as a label pad.

### 4. Preparation of SARS-CoV-2 Test Device

The anti-N protein antibody-immobilized membrane prepared in 1, and the label pad prepared in 2, and 3. were laminated with other members (backing sheet, absorption pad, sample pad) and cut into a width of 5 mm to obtain a SARS-CoV-2 test device.

### 5. Confirmation of Specificity and Accuracy of SARS-CoV-2 Test Device

50 μL of a buffer (10 mM Tris (pH 7.0), 1% (w/v) polyoxyethylene octylphenyl ether, 3% (w/v) arginine, 3% (w/v) BSA) containing a virus that causes respiratory tract infection was dropped onto the SARS-CoV-2 test device prepared in 4, and the device was allowed to stand for 8 minutes.

The result was determined to be positive when color development was visually confirmed at both the application positions of the anti-mouse IgG antibody and the anti-N protein antibody. The result was determined to be negative when color development was visually confirmed only at the application position of the anti-mouse IgG antibody and color development could not be visually confirmed at the application position of the anti-N protein antibody. Some of the determination results for each combination of monoclonal antibodies are shown in Table 2. Positive was judged as 3, 2, or 1 in descending order of color intensity, and negative was judged as 0.

TABLE 2

| | | Anti-N protein antibody-immobilized membrane | | | | | | Monoclonal Antibody No. | | | | | |
|---|---|---|---|---|---|---|---|---|---|---|---|---|---|
| | | A4 | A9 | A12 | A15 | A18 | A20 | A22 | A24 | A29 | A30 | A34 | A38 |
| Anti-N protein | A4 | 0 | 0 | 0 | 0 | 0 | 0 | 0 | 0 | 1 | 2 | 2 | 0 |
| antibody- | A9 | 0 | 0 | 0 | 0 | 0 | 0 | 0 | 0 | 2 | 3 | 2 | 0 |
| immobilized | A12 | 0 | 0 | 0 | 0 | 0 | 0 | 0 | 0 | 1 | 2 | 3 | 0 |
| particles | A15 | 0 | 0 | 0 | 0 | 0 | 0 | 0 | 0 | 2 | 3 | 3 | 0 |
| | A18 | 0 | 0 | 0 | 0 | 0 | 0 | 0 | 0 | 2 | 3 | 3 | 0 |
| | A20 | 0 | 0 | 0 | 0 | 0 | 0 | 0 | 0 | 1 | 2 | 3 | 0 |
| Monoclonal | A22 | 0 | 0 | 0 | 0 | 0 | 0 | 0 | 0 | 2 | 3 | 3 | 0 |
| Antibody No. | A24 | 0 | 0 | 0 | 0 | 0 | 0 | 0 | 0 | 1 | 2 | 3 | 0 |
| | A29 | 2 | 2 | 2 | 2 | 2 | 2 | 3 | 2 | 1 | 2 | 3 | 3 |
| | A30 | 3 | 2 | 2 | 2 | 2 | 3 | 2 | 3 | 2 | 2 | 3 | 2 |
| | A34 | 2 | 3 | 2 | 3 | 3 | 3 | 3 | 3 | 2 | 3 | 3 | 1 |
| | A38 | 0 | 0 | 0 | 0 | 0 | 0 | 0 | 0 | 2 | 3 | 2 | 0 |

As shown in Table 2, when a monoclonal antibody that recognizes the above-mentioned specific region in the N protein (A29, A30, A34) was used for either one or both of the anti-N protein antibody-immobilized membrane and the anti-N protein antibody-immobilized particles, the result was judged as positive, whereas when a monoclonal antibody that does not recognize the Dimerization domain (A4, A9, A12, A15, A18, A20, A22, A24) was used for either one or both of the anti-N protein antibody-immobilized membrane and the anti-N protein antibody-immobilized particles, the result was judged as negative.

1. A method of detecting SARS-CoV-2, comprising detecting SARS-CoV-2 in a sample by an immunoassay method using a monoclonal antibody that specifically reacts with 301aa-319aa, 301aa-320aa, or 351aa-375aa of the amino acid sequence of the N protein of SARS-CoV-2, or an antigen-binding fragment thereof.

2. The method according to claim 1, wherein the immunoassay method is an immunochromatography method.

3. A detection kit for SARS-CoV-2, comprising a monoclonal antibody that specifically reacts with 301aa-319aa, 301aa-320aa, or 351aa-375aa of the N protein of SARS-CoV-2, or an antigen-binding fragment thereof.

4. The detection kit according to claim 3, which is an immunochromatographic test strip.


SEQUENCE LISTING (1 sequence)

SEQ ID NO: 1 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA